(19) United States
(12) Patent Application Publication
(10) Pub. No.: US 2002/0124849 A1
(43) Pub. Date: Sep. 12, 2002

(54) Nasal breathing mask with adjustable thermistor for treating respiratory disorders of sleep

(75) Inventors: Pierre Billette De Villemeur, Le Chesnay (FR); Stephane Thouvier, Argenteuil (FR)

Correspondence Address: YOUNG & THOMPSON, 745 SOUTH 23RD STREET 2ND FLOOR, ARLINGTON, VA, 22202

(73) Assignee: TAEMA

(21) Appl. No.: 09/865,542

(22) Filed: May 29, 2001

(30) Foreign Application Priority Data
May 26, 2000 (FR) 0006773

(51) Int. Cl. A62B 18/02; A62B 7/00; A61M 15/08

(52) U.S. Cl. 128/206.21; 128/207.18

(57) ABSTRACT

A facial respiratory interface (1, 11) comprising an interface body (2, 12) which bears at least one thermistor (3) having an active end (4) which can be placed opposite the mouth of a user when the interface (1, 11) is put into position on the facial region of said user, wherein the length (L) separating the active end (4) from the interface body (2, 12) can be adjusted. The interface can be used for treating respiratory disorders in a patient, in particular sleep apnea.

The present invention concerns a facial respiratory interface, such as a nasal breathing mask or breathing clips, which can be used to diagnose, treat or prevent respiratory disorders in a user, for example sleep apnea.

At present, breathing masks or other facial respiratory interfaces are commonly used for varied and diverse purposes, in particular for administering oxygen, or oxygen-enriched air mixtures, to persons suffering from pulmonary problems; for administering anesthetic gases during the preoperative phase; for administering pressurized air to persons suffering from respiratory disorders, for example sleep apnea; and in the context of treatment using CPAP (continuous positive airway pressure) or treatment using two pressure levels.

In other words, breathing masks and clips make it possible to provide an interface between the barometric and/or volumetric medical ventilation apparatus and the patients who are to be supplied with respiratory gas.

Breathing masks are normally made by molding plastics and are shaped to conform to the contours of the nasal region of the user.

There are in fact two main types of nasal breathing masks, namely those covering the nasal region, and those comprising attachments which are inserted into the nostrils in the manner of cannulas.

These two types of masks in fact differ in principle in the way in which they are rendered leaktight. Thus, in the former case the leaktightness is obtained around the nose, while in the second case the leaktightness is obtained at each nostril.

Masks with nasal attachments have the advantage of avoiding any contact with the bridge of the patient's nose, which bridge is a zone which is usually very sensitive to the rubbing action of industrial masks. However,-masks of this type are difficult to maintain in the correct position on account of the small surface area of contact with the face, which surface area is limited to part of the inner region of the nostrils.

Moreover, the masks covering the nose are the ones most used. These are generally made of a polymer material such as a vinyl chloride or silicone, given that with these materials it is possible to avoid or minimize the irritant effects of the mask on the patient's skin.

The site of delivery of gas can vary depending on the mask, but the majority of them include a central gas delivery orifice whose attachment to the gas delivery circuit of the respiratory assistance apparatus or of the pressurized gas source is obtained with the aid of a connector or similar device. On this point, reference may be made to documents U.S. Pat. Nos. 4,655,213, 3,580,051 and 5,117,819.

Sometimes the mask can include a plurality of gas delivery orifices, as is described in the document U.S. Pat. No. 4,944,310.

In addition, in order to obtain better leaktightness of the mask when the latter is in position on the user's face, the document EP-A-0462701 proposes a nasal mask comprising a shell which is designed to cover the nasal region of the user, on which shell a membrane of an elastomeric material is mounted which is able to distend to define a chamber having an externally convex end region with a thin wall which can inflate outward when the mask is in use, that is to say when it is placed in position on the user's nose.

An alternative to this type of mask is proposed by the documents WO-A-97/09090 which describes a mask which includes a shell intended to receive the nasal region of the user, the leaktightness between said shell and the user's face being obtained by virtue of the presence, on the contour of the shell in contact with the patient's face, of a peripheral chamber filled with a gel.

Moreover, breathing clips for their part are normally made up of a hollow tubular portion carrying respiratory gas and are equipped with two attachments or cannulas intended to be positioned in the nostrils of the user so as to be able to administer therein gas for respiratory assistance.

Respiratory gas is generally delivered via the clips during the inhalation phases and the exhalation phases; during the exhalation phases the gas being exhaled by the patient at the same time as exhaled gases rich in CO₂ coming from the lungs.

Facial respiratory interfaces are presently being increasingly used due in particular to their efficacy and the generalization of the noninvasive techniques of artificial ventilation for various applications in hospitals or outside of hospitals, for example in home care.

Although designed originally to be simple treatment interfaces intended to apply a defined pressure or flowrate of gas to a patient, these very quickly proved to be useful means or instruments of diagnosis for effectively characterizing a patient's breathing.

To this end, these interfaces were very soon equipped with complementary means of diagnosis, in particular thermistors intended to monitor the nasal and/or oral respiratory flow of the patient.

The thermistors used to date have traditionally consisted of a temperature sensor fixed permanently on the interface.

However, given that each patient has his or her particular morphology, it has been found that the thermistor is not always correctly positioned in relation to the patient's mouth.

This then results in a number of problems, in particular unwanted contact between the thermistor and the patient's mouth, or, conversely, too great a distance between them, each of these eventualities leading to losses of the oral flow signal and consequently to sometimes considerable inaccuracies of measurement, which can lead to incorrect diagnoses.

Moreover, given that the interfaces currently used comprise permanently fixed thermistors, there is also the problem of being able to properly disinfect the thermistor from one patient to the next.

It is an object of the present invention therefore to make available a facial respiratory interface with thermistor which does not have the abovementioned disadvantages and which is easy to produce and to use.

The present invention therefore relates to a facial respiratory interface comprising an interface body which bears at least one thermistor having an active end which can be placed opposite the mouth of a user when the interface is put into position on the facial region of said user, wherein the length L separating the active end from the interface body can be adjusted, in particular by the operator.

In the context of the invention, the expression “active end” is to be understood as that part of the thermistor which is positioned opposite or in line with the user's mouth in such a way as to function as a thermistor by detecting the oral respiratory flows of said user.

Depending on the circumstances, the respiratory interface according to the invention can include one or more of the following characteristics:

said active end is moved away from the interface body by exerting a traction on said end in such a way as to increase the length L;

the interface body comprises a support bearing said thermistor;

the support comprises a hollow elongate part within which the thermistor can slide, preferably a tubular part;

the elongate and hollow support part comprises a longitudinal slit;

the elongate and hollow support part comprises an extension forming a T;

the interface is chosen from the group consisting of breathing masks and breathing clips;

it is connected via a gas channel to an apparatus for artificial ventilation.

According to another aspect, the invention also relates to a respiratory apparatus which can be used to treat or diagnose respiratory disorders of sleep in a person, comprising a facial respiratory interface according to the invention; depending on the circumstances, the apparatus is of the type with one or two pressure levels.

The invention will now be described in greater detail with reference to the attached figures which are given by way of illustration but are nonlimiting.

FIG. 1 shows a general side view of a facial respiratory interface 1, in this case a nasal breathing mask according to the invention, which is intended to be positioned on the nasal region of a user.

More precisely, the nasal mask comprises an interface body 2 or shell defining a nasal chamber, which nasal chamber is shaped to cover at least part of the nasal region of the user, that is to say to cover at least the user's nose. The body 2 includes two lateral extensions 8 which are symmetrical and are intended to bear on part of the user's face when said mask is in position on the nasal region of the user, in such a way as to ensure and improve on the one hand the leaktightness and on the other hand the positional hold of the mask on the face.

The body 2 and the lateral extensions 8 are preferably made as a single molded piece, that is to say as a monobloc component made of polymer material, for example silicone or the like.

In addition, the mask body 2 is equipped with a respiratory gas delivery orifice 9, in the area of which delivery orifice 9 there are arranged means 7 of connection with which it is possible to connect the mask to a source of pressurized gas, such as a ventilator or any other apparatus for respiratory assistance delivering a respiratory gas at one or more pressure levels.

In the present case, the means of connection 7 comprise a connector equipped with a connection ring which has fastening means 16 cooperating with secured straps 17 and/or laces and/or similar means of fixing, to allow the site of fastening of the straps 17 to be modified depending on the morphology of the user, in such a way as to permit positioning and effective holding of the mask on the face. To permit effective holding of the mask on the user's face while at the same time distributing the pressure and thus improving the user comfort, a dimension is chosen of between 1 and 5 centimeters and a width of between 1 and 4 centimeters for each of the lateral extensions 8.

The nasal mask according to the invention can be directly connected to a gas circuit carrying a respiratory flow originating from a source of respiratory gas, for example a device for respiratory assistance, to the airways of a patient.

In addition, it will be seen from FIG. 1 that the mask has in its lower part a support 5 having an elongate tubular shape within which there is a thermistor 3 equipped with an active end 4 intended to be placed opposite the user's mouth in order to perform its function there, that is to say to detect the oral respiratory flows.

In accordance with the invention, the length L can be adjusted as desired in order to place the active part 4 correctly opposite the user's mouth irrespective of the morphology thereof.

The length is adjusted by traction, that is to say by gently pulling the thermistor from one direction or the other depending on whether the aim is to increase or decrease the length L.

To permit rapid removal of the assembly, particularly with a view to effective cleaning, the support part 5 comprises a longitudinal part 6 allowing the thermistor 3 to be easily withdrawn in order to proceed with cleaning it and disinfecting it or, conversely, its reinsertion into the tubular support 5 after cleaning.

In addition, as can be seen, the support 5 comprises an extension 5 forming a “T” with the elongate part of said support.

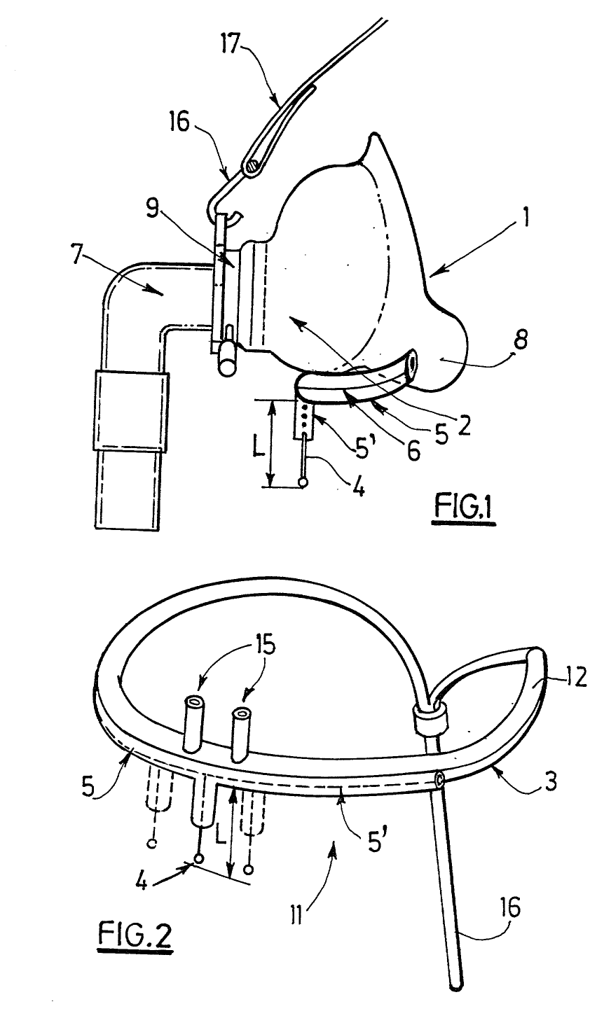

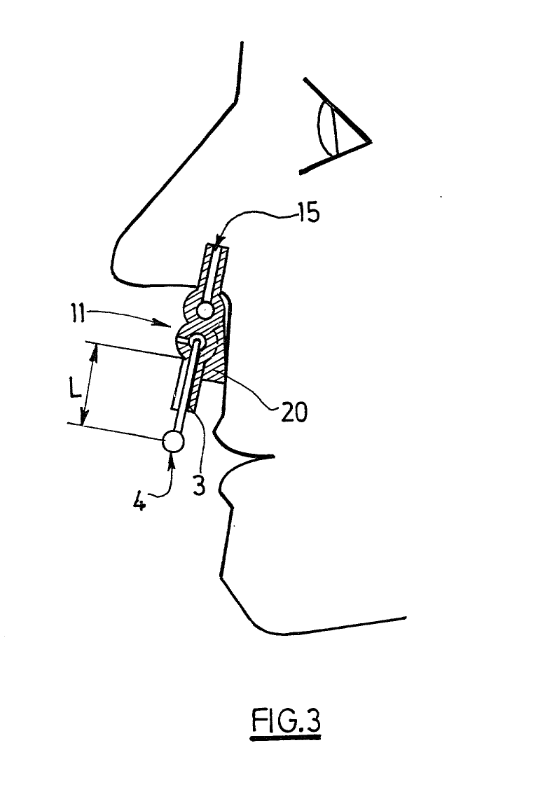

FIGS. 2 and 3 for their part show a second embodiment of a respiratory interface 11 according to the invention, in which the latter is in the form of breathing clips 11.

These breathing clips are made up of a clip body 12 of tubular shape intended to carry the gas to the nostrils of the patient to whom the gas is administered by way of two cannulas 15.

The body 12 has in its lower part a hollow tubular support 5 within which is inserted a thermistor 3 which, as before, has an active end 4 separated from the support by an adjustable distance L; the thermistor 3 once again being movable by sliding in the support 5.

To make it easier to fit and remove the assembly, the support 5 is, as before, slotted 5′ along its entire length.

FIG. 3 shows the clips from FIG. 2 in position on a user's face, and this will clearly show the advantage of being able to vary, that is to say modify or adjust as desired, the distance L in order to correctly place the end 4 of the thermistor 3 opposite the user's mouth.

A respiratory interface according to the present invention can be used for systematic treatment of respiratory disorders of sleep, in particular sleep apnea or snoring.

1. A facial respiratory interface (1, 11) comprising an interface body (2, 12) which bears at least one thermistor (3) having an active end (4) which can be placed opposite the mouth of a user when the interface (1, 11) is put into position on the facial region of said user, wherein the length (L) separating the active end (4) from the interface body (2, 12) can be adjusted.

2. The interface as claimed in claim 1, wherein said active end (4) is moved away from the interface body (2, 12) by exerting a traction on said end in such a way as to increase the length (L).

3. The interface as claimed in either of claims 1 and 2, wherein the interface body (2, 12) comprises a support (5) bearing said thermistor (3).

4. The interface as claimed in one of claims 1 through 3, wherein the support (5) comprises a hollow elongate part within which the thermistor can slide, preferably a tubular part.

5. The interface as claimed in either of claims 3 and 4, wherein the elongate and hollow support part comprises a longitudinal slit (6).

6. The interface as claimed in one of claims 3 through 5, wherein the elongate and hollow support part comprises an extension (5) forming a T.

7. The interface as claimed in one of claims 1 through 6, wherein the interface is chosen from the group consisting of breathing masks and breathing clips.

8. The interface as claimed in one of claims 1 through 7, wherein it is connected via a gas channel to an apparatus for artificial ventilation.

9. A respiratory apparatus which can be used to treat or diagnose respiratory disorders of sleep in a person, comprising a facial respiratory interface (1, 11) as claimed in one of claims 1 through 8, said interface preferably being connected to said apparatus by way of one or more gas channels.

10. The apparatus as claimed in claim 9, being of the type with one or two pressure levels.
